(19) United States
(12) Patent Application Publication
(10) Pub. No.: US 2022/0273443 A1
(43) Pub. Date: Sep. 1, 2022

(54) HYDROGEL IMPLANT PROCEDURE

(71) Applicant: CARTIVA, INC., Alpharetta, GA (US)

(72) Inventors: Richard Clay HALES, Alpharetta, GA (US); Stephanie Marie MANSUETI, Atlanta, GA (US)

(73) Assignee: CARTIVA, INC., Alpharetta, GA (US)

(21) Appl. No.: 17/633,770

(22) PCT Filed: Aug. 19, 2020
(86) PCT No.: PCT/US2020/046876

§ 371 Date: Feb. 8, 2022

Related U.S. Application Data
(60) Provisional application No. 62/900,704, filed on Sep. 16, 2019.

(51) Int. Cl. A61F 2/30; A61F 2/46; A61L 27/52

(52) U.S. Cl. CPC A61F 2/30756; A61F 2/4618; A61L 27/52; A61F 2002/30761; A61F 2002/30784; A61F 2002/30322; A61B 17/14

(57) ABSTRACT

Surgical procedures for repairing articulating surface in a joint using implants such as hydrogel implants are disclosed.

### CROSS-REFERENCE TO RELATED APPLICATIONS

This application claims the benefit of U.S. Provisional Application Ser. No. 62/900,704, filed on Sep. 16, 2019, entitled “HYDROGEL IMPLANT PROCEDURE,” the disclosure of which is incorporated herein by reference in its entirety.

### FIELD OF DISCLOSURE

The present disclosure relates generally to orthopedic surgical procedures, and more specifically, to procedures for repairing articulating surface in a joint using implants such as hydrogel implants.

### BACKGROUND

Implants can be used to replace deteriorated or otherwise damaged articulating surface (i.e., the cartilage tissue) within a joint. Such devices can be used to treat osteoarthritis, rheumatoid arthritis, other inflammatory diseases, generalized joint pain, and joint damages.

In order to install a cartilage surface repairing implant in a joint, the cartilage surface of a bone in the joint is resected with appropriate cuts to prepare the bone surface for receiving the implant. In order to resect the bone surface in the joint with accuracy and proper orientation, instruments for guiding the cutting tools are utilized to aid the surgeon so that such procedure can be performed repeatedly and with accuracy.

### SUMMARY

A method for repairing a damaged portion of an articulating surface of a joint according to an embodiment is disclosed. The method comprising:

providing an implant that comprises:

a main portion configured for inserting into a joint, wherein the main portion comprises:
a porous material portion having a first bone-engaging surface having a first contour; and
a hydrogel portion that is bonded to the porous material portion and forming an articulating surface opposite from the first bone-engaging surface; and

a bone plate portion configured for securing the implant to a bone that forms the joint;
wherein, the main portion having a leading end and a trailing end, wherein the leading end is configured for being inserted into the joint;
wherein the bone plate portion is integrally formed with the porous material portion and extends from the trailing end, forming a second bone-engaging surface that is also formed of the porous material and extends from the first bone-engaging surface in a direction opposite from the articulating surface at an angle with respect to the first bone-engaging surface, wherein the second bone-engaging surface has a second contour;
wherein the bone plate portion comprises a solid metal portion that forms all exterior surfaces of the bone plate portion except for the second bone-engaging surface; and
wherein the bone plate portion has at least one screw hole for receiving a bone screw;
   preparing the damaged portion of the articulating surface of a bone in the joint to have two resected surfaces, one resected surface with a contour that matches the first contour of the first bone-contacting surface, and the second resected surface with a contour that matches the second contour of the second bone-contacting surface of the implant; and implanting the implant into the joint.

A method for repairing a damaged portion of an articulating surface of a joint according to another embodiment is disclosed. The method comprising:

providing an implant that comprises:

a main portion configured for inserting into the joint, wherein the main portion comprises:
a hydrogel portion forming a bone-contacting surface and an articulating surface opposite from the bone-contacting surface;
wherein the bone-contacting surface has a contour that comprises a protruding part that has a half-cylinder contour;
wherein, the main portion having a leading end and a trailing end, wherein the leading end is configured for being inserted into the joint; and

a bone plate portion configured for securing the implant to a bone that forms the joint; wherein the bone plate portion comprises:
a first part having a perforated structure that is embedded in the protruding part of the hydrogel portion; and
a second part that is not embedded in the protruding part of the hydrogel portion and extending from the trailing end in a direction opposite from the articulating surface at an angle ≤160° but ≥80° with respect to the bone-contacting surface;
wherein the second part has at least one screw hole for receiving a bone screw; preparing the damaged portion of the articulating surface of the joint to have a resected surface with a contour that matches the contour of the bone-contacting surface of the implant; and

implanting the implant into the joint.

A method for repairing a damaged portion of an articulating surface of a joint according to another embodiment is disclosed. The method comprising:

providing an implant that comprises:

a main portion configured for inserting into the joint and comprising a leading end, a trailing end, an articulating surface and a bone-contacting surface extending between the leading end and the trailing end, wherein the bone-contacting surface has a contour, wherein the leading end is configured for being inserted into the joint, wherein the main portion further comprises:
a hydrogel portion forming the articulating surface and the bone-contacting surface opposite from the articulating surface; and
a porous material portion bonded to the hydrogel portion and forming a protruding part having a half-cylinder shape that extends from the trailing end and partially towards the leading end, the protruding part forming a portion of the bone-contacting surface;
wherein the porous material portion comprises a tapered hole at the trailing end; and

a bone plate configured for securing the implant to a bone that forms the joint;
wherein the bone plate is formed of a solid metal;
wherein the bone plate comprises a tapered stem that is configured to be inserted into the tapered hole in the porous material portion, whereby the tapered stem and the tapered hole cooperate to urge the bone-contacting surface of the implant toward the bone when the implant is inserted into the joint; and

wherein the bone plate has at least one screw hole for receiving a bone screw; preparing the damaged portion of the articulating surface of the joint to have a resected surface with a contour that matches the contour of the bone-contacting surface of the implant; and implanting the implant into the joint.

### BRIEF DESCRIPTION OF THE DRAWINGS

The various embodiments of the inventive subject matter of the present disclosure will be described in more detail in conjunction with the following drawing figures. The structures in the drawing figures are illustrated schematically, and they are not drawn to scale. The drawings figures are not intended to show actual dimensions.

FIG. 1 is a flowchart for the method for repairing a damaged portion of an articulating surface of a bone forming a joint according to an embodiment.

FIG. 2 is a perspective view illustration of a hydrogel implant according to a first embodiment of the present disclosure.

FIG. 3 is an exploded view illustration of the hydrogel implant of FIG. 2.

FIG. 4A is a side view illustration of the hydrogel implant of FIG. 2.

FIG. 4B is a side view illustration of the hydrogel implant of FIG. 2 in implanted position in a joint.

FIG. 5 is a cross-section view illustration of the hydrogel implant of FIG. 2 taken through the section line H-H shown in FIG. 3A.

FIG. 6 is a top view illustration of the hydrogel implant of FIG. 2 showing only the porous metal foam portion, i.e., the implant without the hydrogel portion and the solid metal portion.

FIG. 7 is a cross-section view illustration of the structure shown in FIG. 6 taken through the section line T-T.

FIG. 8 is a side view illustration of the hydrogel implant of FIG. 2 showing only the porous metal foam and the solid metal portions, i.e., the implant without the hydrogel portion.

FIG. 9 is a cross-section view illustration of the structure shown in FIG. 8 taken through the section line E-E.

FIG. 10 is a top view illustration of the structure shown in FIG. 8.

FIG. 11 is a top view illustration of the hydrogel implant of FIG. 2.

FIG. 12 is a cross-section view illustration of the hydrogel implant taken through the section line M-M shown in FIG. 11.

FIG. 13 is a detailed view of the region N identified in the sectional view of FIG. 12.

FIGS. 14 and 15 are illustrations showing the resected bone surface B1.

FIG. 16 is a flowchart for the method for repairing a damaged portion of an articulating surface of a bone forming a joint according to an embodiment.

FIG. 17 is a perspective view of a hydrogel implant according to a second embodiment.

FIG. 18 is an exploded view illustration of the hydrogel implant of FIG. 17.

FIG. 19 is a top view of the bone plate portion of the hydrogel implant of FIG. 17.

FIG. 20 is a side view of the bone plate portion shown in FIG. 19.

FIG. 21 is a cross-section view of the bone plate portion taken through the section line F-F shown in FIG. 19.

FIG. 22 is a detailed view of the region G identified in the sectional view of FIG. 21.

FIG. 23 is a detailed view of the region N identified in the sectional view of FIG. 21.

FIG. 24 is an illustration showing the resected bone surface B3.

FIG. 25 is a flowchart for the method for repairing a damaged portion of an articulating surface of a bone forming a joint according to an embodiment.

FIG. 26A is a perspective view of a hydrogel implant according to another embodiment.

FIG. 26B is an exploded view illustration of the hydrogel implant of FIG. 26A.

FIG. 27 is a top view of the bone plate portion of the hydrogel implant of FIG. 26A.

FIG. 28 is a cross-section view of the bone plate portion taken through the section line C-C shown in FIG. 27.

FIG. 29 is a detailed view of the region E identified in the sectional view of FIG. 28.

FIG. 30 is an illustration showing the resected bone surfaces B3 and B4.

FIG. 31 is a perspective view illustration of a hydrogel implant according to another embodiment.

FIGS. 32-33 are illustrations showing the hydrogel implant of FIG. 31 implanted into some of the mid-foot joints.

FIG. 34 is a flowchart for the method for repairing a damaged portion of an articulating surface of a bone forming a joint according to an embodiment.

FIG. 35 is a perspective view illustration of the main portion of the hydrogel implant of FIG. 31.

FIG. 36 is an illustration showing a view of the bone-contacting surface of the main portion shown in FIG. 35.

FIG. 37 is a top view illustration of the main portion shown in FIG. 35.

FIG. 38 is a side view illustration of the main portion shown in FIG. 35.

FIG. 39 is a cross-section view of the main portion shown in FIG. 35 taken through the section line A-A shown in FIG. 37.

FIG. 40 is a perspective view of the hydrogel implant of FIG. 31 secured to a bone in its fully implanted configuration.

FIG. 41 is a cross-section view of the hydrogel implant shown in FIG. 40 in which the bone plate portion has not yet been seated in its fully implanted configuration.

FIG. 42 is a cross-section view of the hydrogel implant shown in FIG. 40 in which the bone plate portion is seated in its fully implanted configuration.

### DETAILED DESCRIPTION

This description of the exemplary embodiments is intended to be read in connection with the accompanying drawings, which are to be considered part of the entire written description. The drawing figures are not necessarily to scale, and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

As used herein, “bone cutting” encompasses sawing of bones as well as removing or grinding bone material using a burr.

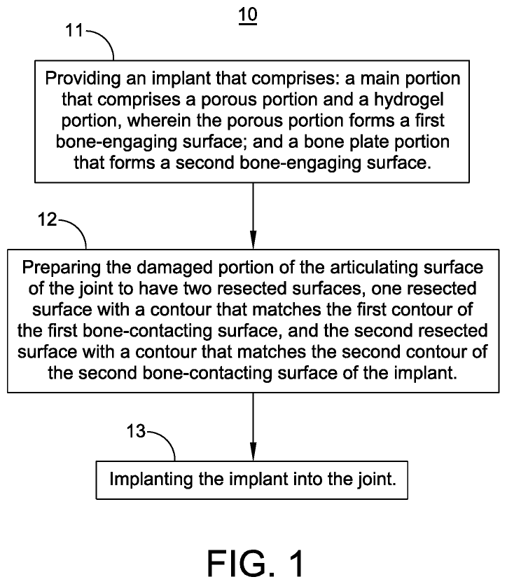

Referring to flowchart 10 in FIG. 1 a method for repairing a damaged portion of an articulating surface of a joint according to an embodiment of the present disclosure is disclosed. Referring to FIG. 1, the method comprises providing an implant 100 that comprises a main portion 110 configured for inserting into a joint and a bone plate portion 120 extending from the main portion 110 at an angle and configured for securing the implant 100 to a bone that forms the joint. See box 11. The implant 100 is configured with a first bone-contacting surface 130 having a first contour, and a second bond-contacting surface 140 having a second contour. The method also comprises: preparing the damaged portion of the articulating surface of a bone in the joint to have two resected surfaces B1, B2, one resected surface B1 with a contour that matches the first contour of the first bone-contacting surface 130, and the second resected surface B2 with a contour that matches the second contour of the second bone-contacting surface 140 of the implant (see box 12); and then implanting the implant 100 into the joint (see box 13). Implanting the implant 100 into the joint comprises securing the implant 100 to the two resected surfaces B1, B2 of the bone.

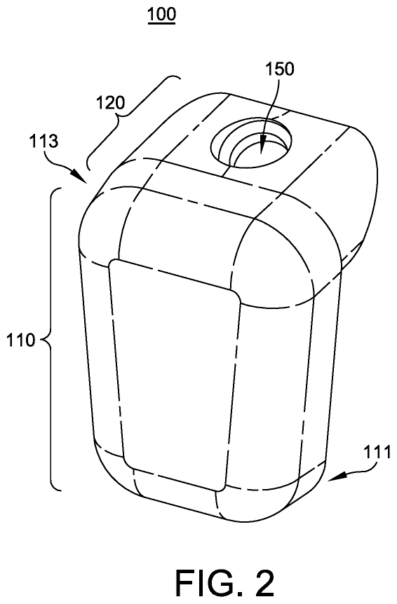

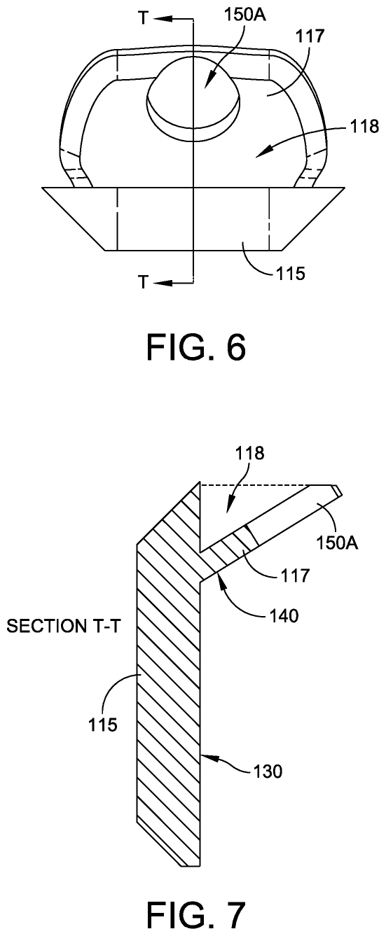

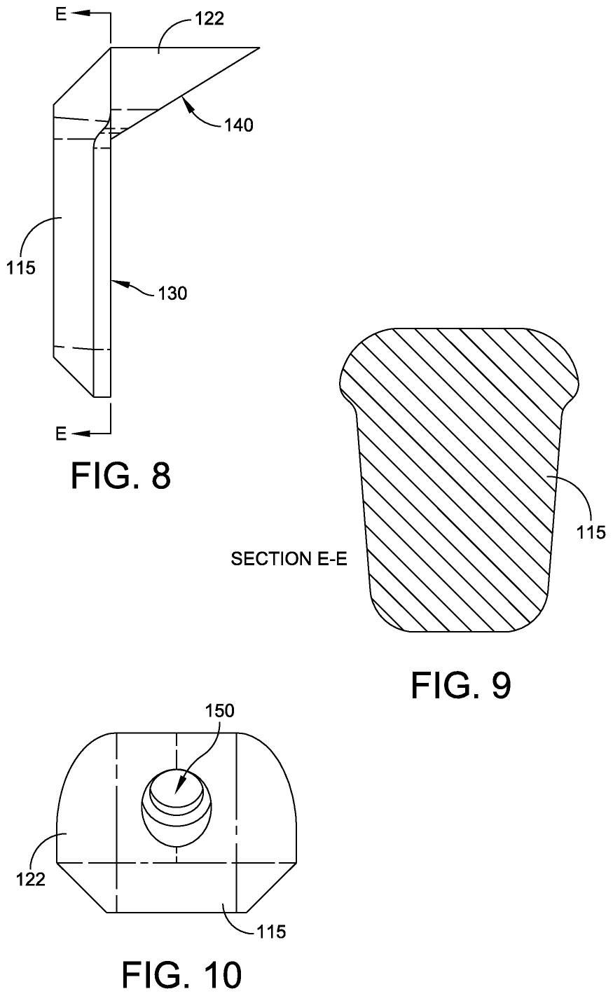

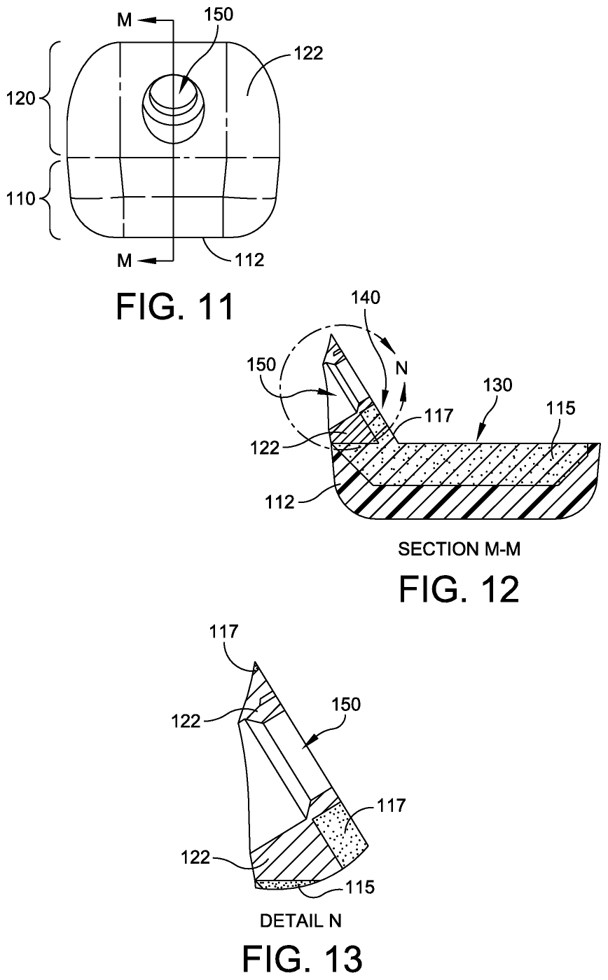

The detailed structure of the implant 100 is illustrated in FIGS. 2-13. The main portion 110 comprises a porous material portion 115 having the first bone-engaging surface 130, having the first contour, and a hydrogel portion 112 that is bonded to the porous material portion 115 and forming an articulating surface 114 located opposite from the first bone-engaging surface 130. The bone plate portion 120 is configured for securing the implant 100 to a bone that forms the joint. The main portion 110 comprises a leading end 111 and a trailing end 113, wherein the leading end is configured for being inserted into the joint. Here, the terms “leading” and “trailing” references generally the implant's orientation in its implanted position in a joint space and also the orientation as the implant is being inserted into the joint space.

The bone plate portion 120 is integrally formed with the porous material portion 115 and extends from the trailing end 113, forming the second bone-engaging surface 140 that is also formed of the porous material and extends from the first bone-engaging surface 130 in a direction opposite from the articulating surface 114 at an angle θ with respect to the first bone-engaging surface 130. The second bone-engaging surface 140 has the second contour.

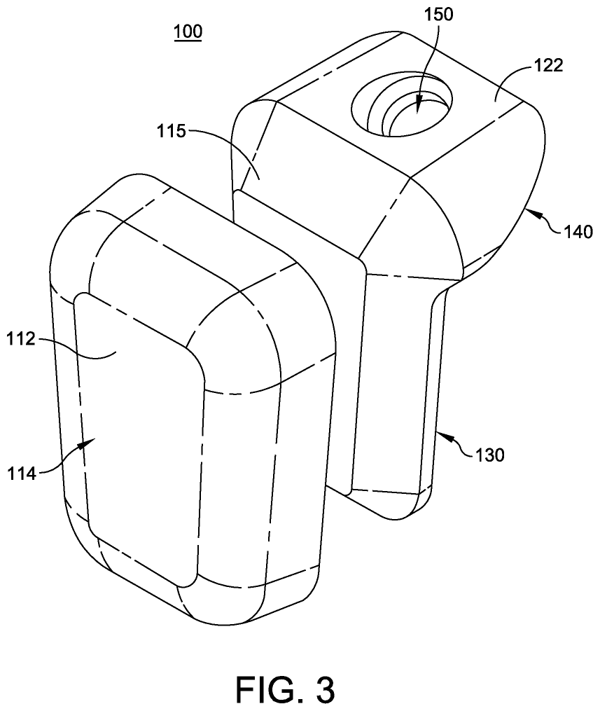

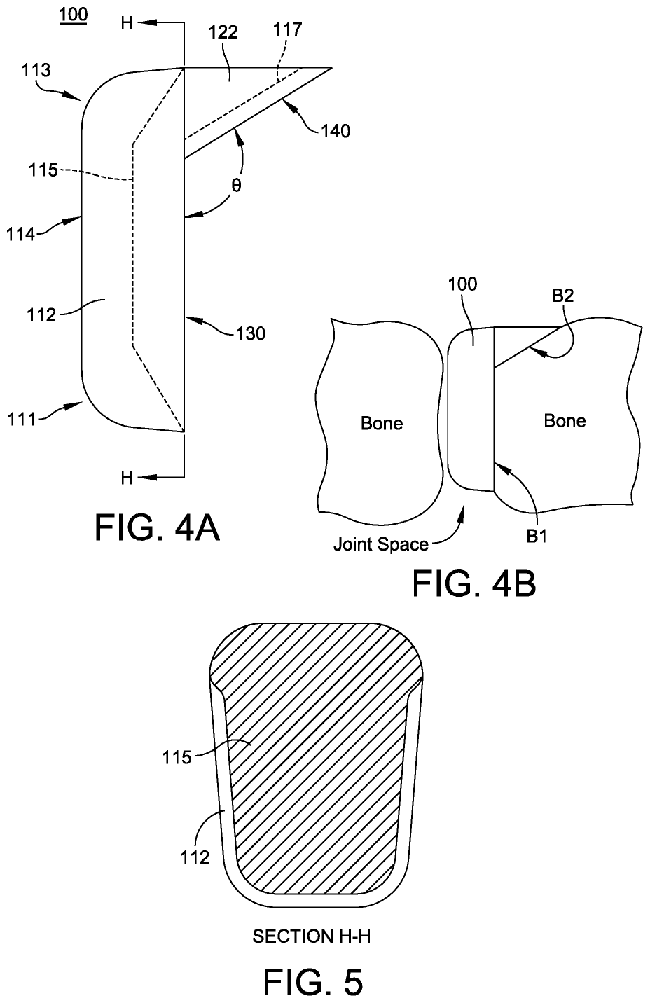

Referring to FIGS. 3 and 4A, the hydrogel portion 112 forms an articulating surface 114 located opposite from the first bone-engaging surface 130. In other words the articulating surface 114 and the first bone-engaging surface 130 face away from each other. The bone plate portion 120 comprises a solid metal portion 122 that forms all exterior surfaces of the bone plate portion 120 except for the second bone-engaging surface 140. The second bone-engaging surface 140 of the bone plate portion 120 is formed of the same porous material as the porous material portion 115 and is preferably integrally formed with the porous material portion 115 as a unitary structure for ease of manufacturing and producing a more compact structure.

The bone plate portion 120 comprises at least one screw hole 150 for receiving a bone screw that is used to secure the implant 100 to a bone. There can be more than one screw hole provided in the bone plate portion 120 for implanting into a joint repair site that may require more than one bone screw to secure the implant.

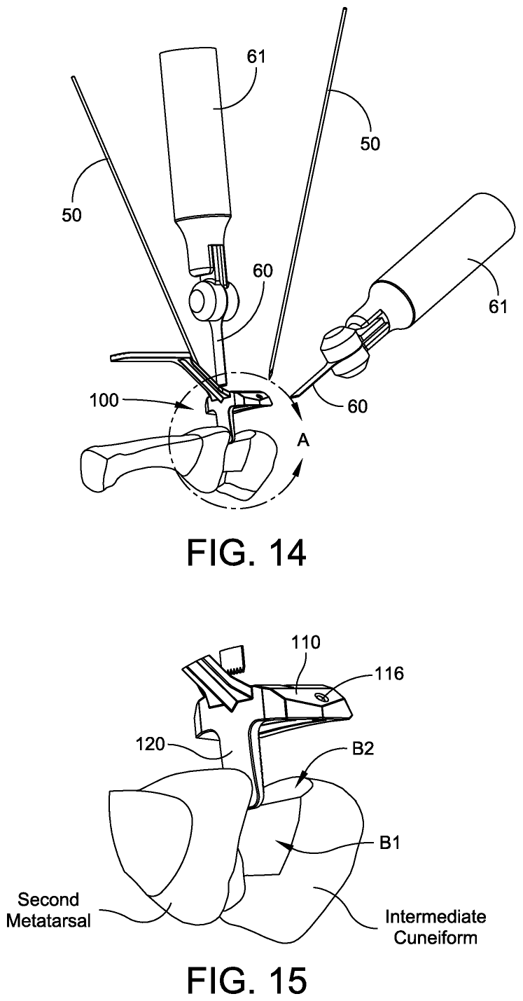

Referring to FIGS. 14-15, the preparation of the two resected surfaces B1, B2 according to some embodiments will be described. The step of preparing the damaged portion of the articulating surface of the joint comprises: cutting into the articulating surface of the joint with a bone saw 60 at a first angle to remove a portion of the articulating surface and form a first resected surface B1 that has a contour that matches the first contour of the first-bone engaging surface 130; and then cutting into the articulating surface of the joint with a bone saw 60 at a second angle to remove a portion of the articulating surface and form a second resected surface B2 that has a contour that matches the second contour of the second-bone engaging surface 140. In the illustrated example, the joint being repaired is a second TMT joint and the bone whose cartilage surface is being repaired is the intermediate cuneiform. As shown in FIG. 15, the resected surfaces B1 and B2 are flat surfaces.

Using the illustrations of FIGS. 14-15 as a reference, in some embodiments, the step of preparing the damaged portion of the articulating surface of the joint comprises: cutting into the articulating surface of the joint with a bone saw 60 at a first angle to remove a portion of the articulating surface and form a first resected surface B2 that has a contour that matches the second contour of the second-bone engaging surface 140; and then cutting into the articulating surface of the joint with a bone saw 60 at a second angle to remove a portion of the articulating surface and form a second resected surface B1 that has a contour that matches the first contour of the first-bone engaging surface 130. As illustrated, it is generally desired to use some sort of a cutting guide 100 to guide that comprise appropriate guide slots that guide the motion and direction of the cutting tool (the bone saw 60 in this example) during the cutting procedure. In other embodiments, the cutting tool can be a rotating burr rather than the bone saw 60. When a cutting tool such as a rotating burr is used, the associated cutting guide would have guiding slots that can accommodate the width of the burr.

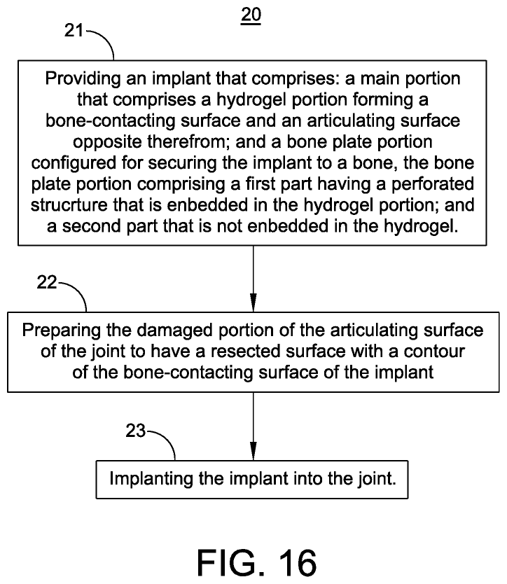

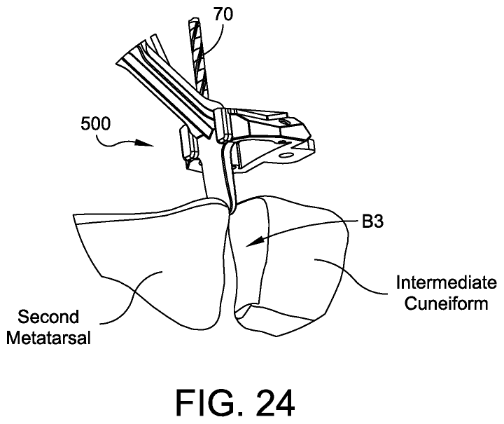

Referring to flowchart 20 in FIG. 16 a method for repairing a damaged portion of an articulating surface of a joint according to another embodiment is disclosed. The method comprises: providing an implant 200 that comprises: a main portion that comprises, a hydrogel portion forming a bone-contacting surface and an articulating surface opposite from the bone-contacting surface, and a bone plate portion configured for securing the implant to a bone that forms the joint (see box 21), preparing the damaged portion of the articulating surface of the joint to have a resected surface B3 (see FIG. 24) with a contour that matches the contour of the bone-contacting surface 230 of the implant 200 (see box 22); and implanting the implant 200 into the joint (see box 23). Implanting the implant 200 into the joint comprises securing the implant 200 to the resected surface of the bone.

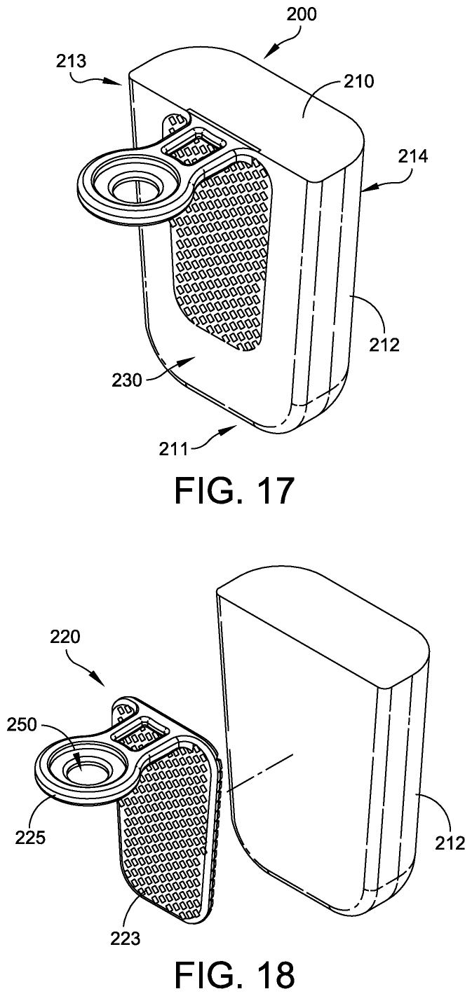

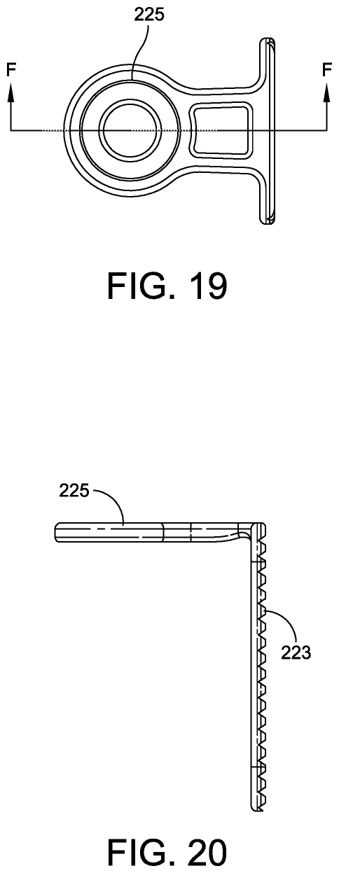

The detailed structure of the implant 200 is illustrated in FIGS. 17-23. The implant 200 comprises a main portion 210 configured for inserting into a joint. The main portion 210 can comprise a hydrogel portion 212 forming the bone-contacting surface 230 and an articulating surface 214 opposite from the bone-contacting surface 230. The main portion 210 has a leading end 211 and a trailing end 213, wherein the leading end is configured for being inserted into the joint. A bone plate portion 220 configured for securing the implant 200 to a bone that forms the joint. The bone plate portion 220 comprises a first part 223 having a perforated structure that is embedded in the hydrogel portion 212; and a second part 225 that is not embedded in the hydrogel portion and extending from the trailing end 213 in a direction opposite from the articulating surface 214 at an angle ≤160° but ≥80° with respect to the bone-contacting surface 230. The second part 225 has at least one screw hole 250 for receiving a bone screw (not shown). The second part 225 can have generally circular configuration around the screw hole 250 as shown in FIG. 15, but the shape of the second part 225 can be designed to have any appropriate shape to fit into the structure (e.g. contour) of the bones around the particular joint space into which the implant 200 will be implanted.

In some embodiments of the implant 200, the second part 225 extends from the trailing end 213 at an angle that is ≤110° and ≥80°. In some embodiments of the implant 200, the second part 225 extends from the trailing end 213 at an angle that is substantially 90° (i.e., 90±2°. In some embodiments of the implant 200, the first part 223 of the bone plate portion 220 is embedded in the hydrogel portion 212 and located closer to the bone-contacting surface 230 than the articulating surface 214. In some embodiments of the implant 200, the contour of the bone-contacting surface 230 is a flat surface. When the bone-contacting surface 230 is a flat surface, the first part 223 of the bone plate portion 220 has substantially flat configuration as shown in FIGS. 13 and 14 to correspond to the flat contour of the bone-contacting surface 230.

The implant 200 can be formed by molding the hydrogel material around the first part 223 of the bone plate portion 220 using injection molding or open cavity molding processes known to those in the art. As shown in FIGS. 18 and 23, the first part 223 of the bone plate portion 220, the part that gets embedded in the hydrogel portion 212, can be perforated with holes 227 to better enable the hydrogel material to intimately surround and envelope the first part 223 during the molding process so that the resulting implant 200 has the optimal structural integrity.

Referring to the illustration in FIG. 24, in some embodiments, the step of preparing the damaged portion of the articulating surface of the joint comprises cutting into the articulating surface of the joint with a cutting tool 70 to remove a portion of the articulating surface and form a flat resected surface B3 that forms at least a portion of the contour of the resected surface. In the illustrated example, the bone whose articulating surface is being repaired is an intermediate cuneiform. As mentioned previously, the procedure for cutting the bone to form the resected surface B3 preferably utilizes a cutting guide 500 to guide the motion and direction of the cutting tool 70 during the cutting procedure. In this illustrated example, the cutting tool 70 is a rotating burr and the cutting guide 500 is configured to guide the rotating burr 70. As mentioned earlier, the cutting tool can be a rotating burr or a bone saw 60.

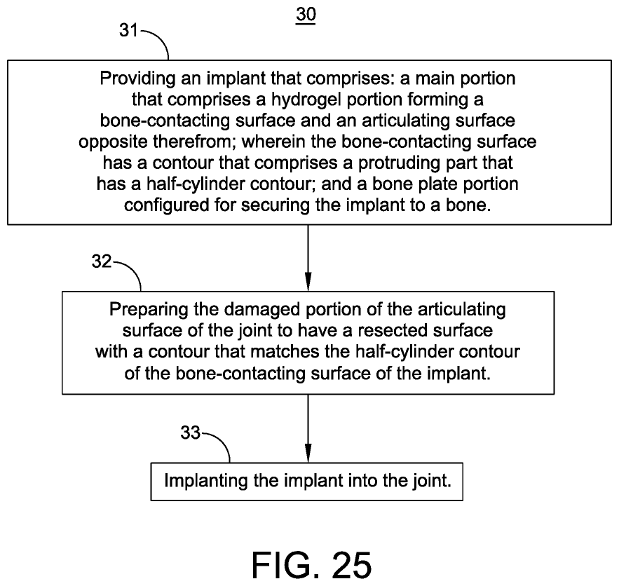

Referring to flowchart 30 in FIG. 25 a method of repairing a damaged portion of an articulating surface of a joint according to another embodiment is disclosed. The method comprises: providing an implant 300 that comprises: a main portion that comprises a hydrogel portion forming a bone-contacting surface and an articulating surface opposite from the bone-contacting surface, wherein the bone-contacting surface has a contour that comprises a protruding part that has a half-cylinder contour; and a bone plate portion configured for securing the implant 300 to the bone that forms the joint (see box 31); preparing the damaged portion of the articulating surface of the joint to have a resected surface B4 with a contour that matches the half-cylinder contour of the bone-contacting surface 330 of the implant 300 (see box 32); and implanting the implant 300 into the joint (see box 33).

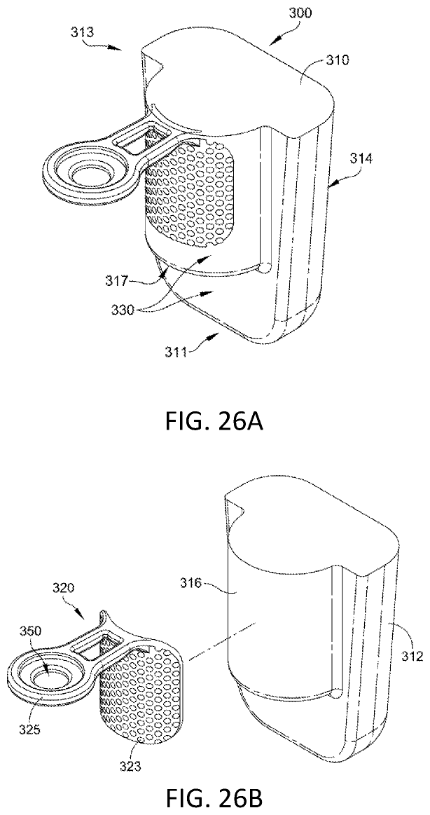

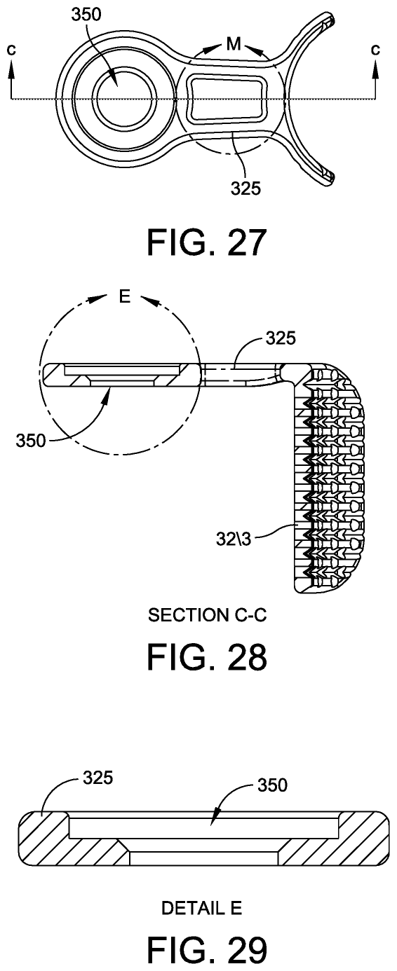

The detailed structure of the implant 300 is illustrated in FIGS. 26-29. The implant 300 is similar to the implant 200 with one of the differences being the provision of a protruding part 316 on the bone-contacting surface 330. The implant 300 for replacing a portion of an articulating surface of a joint comprises a main portion 310 configured for inserting into a joint. The main portion 310 comprises a hydrogel portion 312 forming a bone-contacting surface 330 and an articulating surface 314 opposite from the bone-contacting surface 330. The bone-contacting surface 330 comprises the protruding part 316 that provides additional structural stability at the interface between the bone and the bone-contacting surface 330 when the implant 300 is implanted in position in a joint space. Preferably, the bone surface that is receiving the implant 300 would be prepared to have a contour that is complementary to the contour of the bone-contacting surface 330 that includes the protruding part 316.

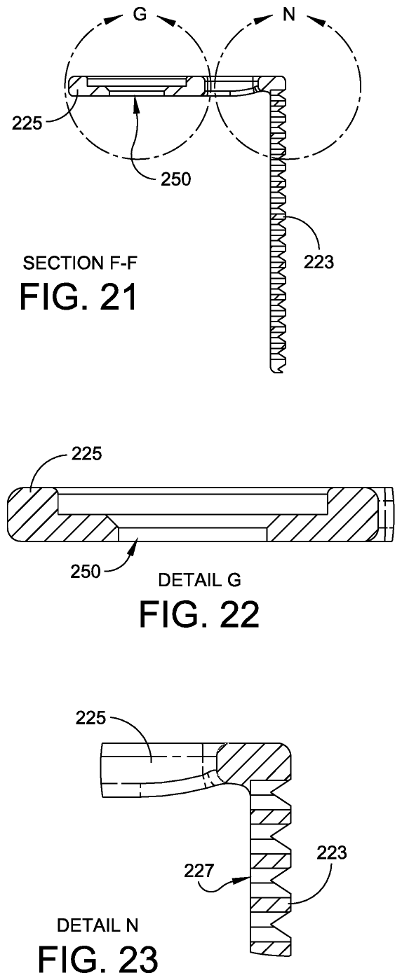

Similar to the implant 200, the main portion 310 of the implant 300 comprises a leading end 311 and a trailing end 313, where the leading end 311 is configured for being inserted into the joint. The implant 300 further comprises a bone plate portion 320 configured for securing the implant 300 to a bone that forms the joint. The bone plate portion 320 comprises a first part 323 having a perforated structure that is embedded in the protruding part 316 of the hydrogel portion 312, and a second part 325 that is not embedded in the protruding part of the hydrogel portion. The second part 325 extends from the trailing end 313 in a direction opposite from the articulating surface 314 at an angle ≤160° but ≥80° with respect to the base flat portion of the bone-contacting surface 330 (i.e., the part of the bone-contacting surface 330 excluding the protruding part 316. The second part has at least one screw hole 350 for receiving a bone screw (not shown). Similar to the implant 200, the second part 325 can have generally circular configuration around the screw hole 350 as shown in FIG. 21, but the shape of the second part 325 can be designed to have any appropriate shape to fit into the structure (e.g. contour) of the bones around the particular joint space into which the implant 300 will be implanted.

In some embodiments of the implant 300, the second part 325 extends from the trailing end 313 at an angle that is ≤110° and >80°. In some embodiments of the implant 300, the second part 325 extends from the trailing end 313 at an angle that is substantially 90° (i.e., 90±2°. In some embodiments, the first part 323 of the bone plate portion is embedded in the hydrogel portion and located closer to the bone-contacting surface 330 than the articulating surface 314. Preferably, the first part 323 of the bone plate portion 320 has a contour that substantially matches the contour of the protruding part 316 of the hydrogel portion 312.

In some embodiments, the protruding part 316 of the bone-contacting surface 330 has a half-cylinder contour and the first part 323 of the bone plate portion has a complementary curved contour.

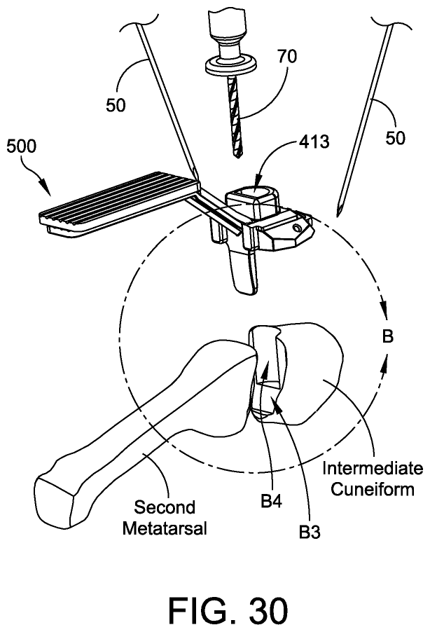

Referring to the illustration in FIG. 30, in some embodiments, the step of preparing the damaged portion of the articulating surface of the joint to have a resected surface with a contour that matches the half-cylinder contour of the bone-contacting surface of the implant 300 comprises cutting into the articulating surface of the bone that forms the joint with a cutting tool 70 to remove a portion of the articulating surface and form a half-cylinder contoured resected surface B4. In this example, the cutting tool 70 is a rotating burr and the cutting guide 500 comprises a guide slot 413 that has a half-cylinder shaped opening to guide the cutting tool 70 in forming the half-cylinder shaped cut into the bone. In this example the joint being repaired is a second TMT joint and the bone whose distal end is being resected is the intermediate cuneiform. The resected bone surface B4 has the half-cylinder contour that matches the contour of the protrusion 316 in the bone-contacting surface of the implant 300.

In some embodiments, the step of preparing the damaged portion of the articulating surface comprises cutting a flat resected surface B3 first, then the subsequent step of cutting into the flat resected surface B3 of the articulating surface with a burr 70 to form the half-cylinder contoured surface B4.

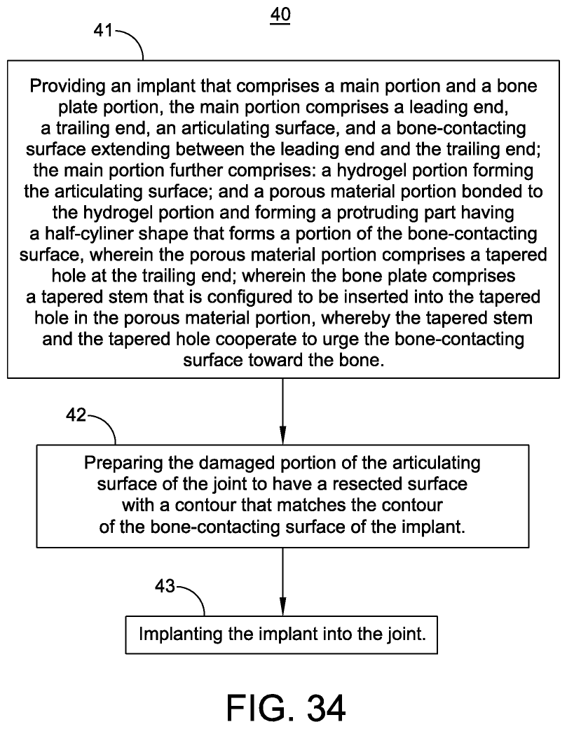

Referring to flowchart 40 in FIG. 34 a method for repairing a damaged portion of an articulating surface of a joint according to another embodiment is disclosed. The method comprises: providing an implant 400, preparing the damaged portion of the articulating surface of the joint to have a resected surface B4 with a contour that matches the contour of the bone-contacting surface 430 of the implant 400 (see box 42); and implanting the implant 400 into the joint (see box 43).

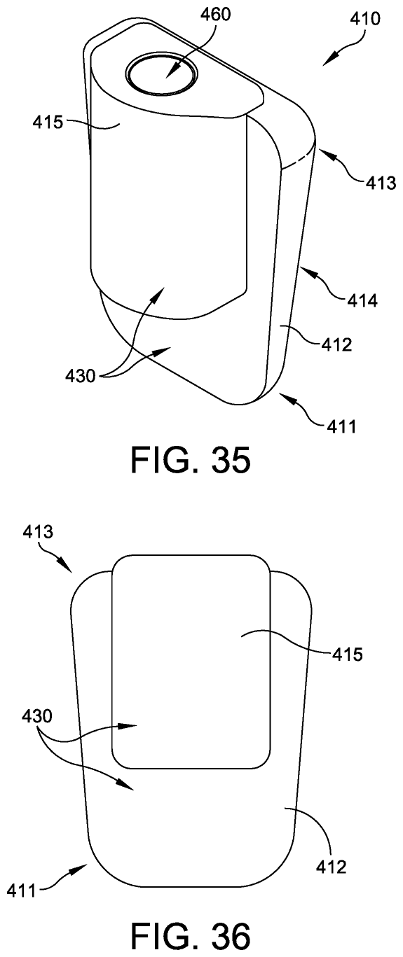

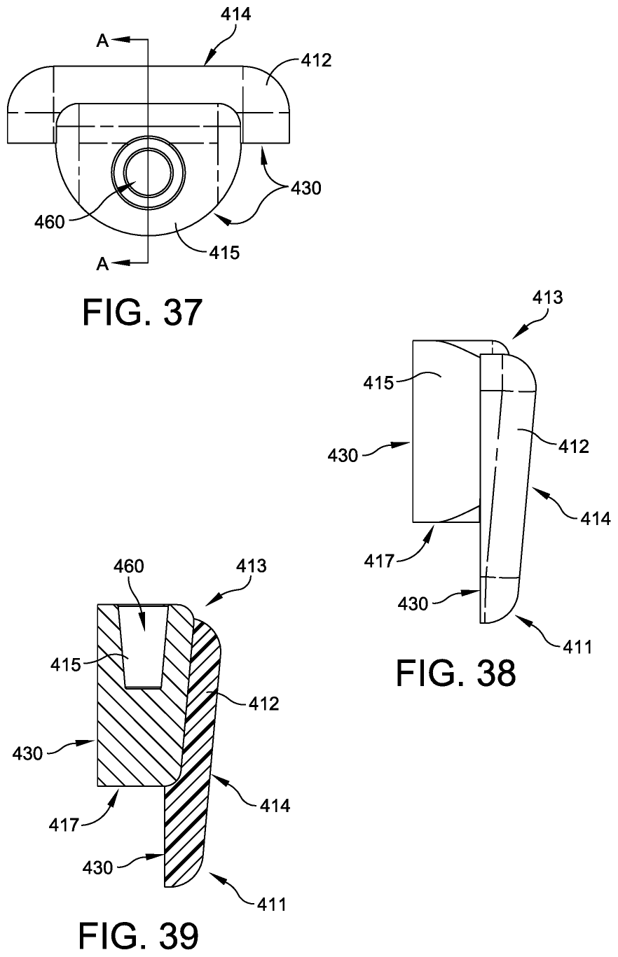

The detailed structure of the implant 400 is illustrated in FIGS. 35-42. The implant 400 comprises a main portion 410 configured for inserting into a joint and a bone plate 420 configured for securing the implant 400 to a bone that forms a joint.

Referring to FIG. 35, the main portion 410 comprises a leading end 411, a trailing end 413, an articulating surface 414 and a bone-contacting surface 430 extending between the leading end and the trailing end. The leading end 411 is configured for being inserted into the joint. The main portion 410 further comprises a porous material portion 415 and a hydrogel portion 412 forming the articulating surface 414 and the bone-contacting surface 430 opposite from the articulating surface 414. The porous material portion 415 is bonded to the hydrogel portion 412 and the porous material portion 415 extends partially from the trailing end 413 towards the leading end 411 and forms a portion of the bone-contacting surface 430.

Referring to FIG. 35 and the cross-sectional view in FIG. 39, the porous material portion 415 comprises a tapered hole 460 at the trailing end 413.

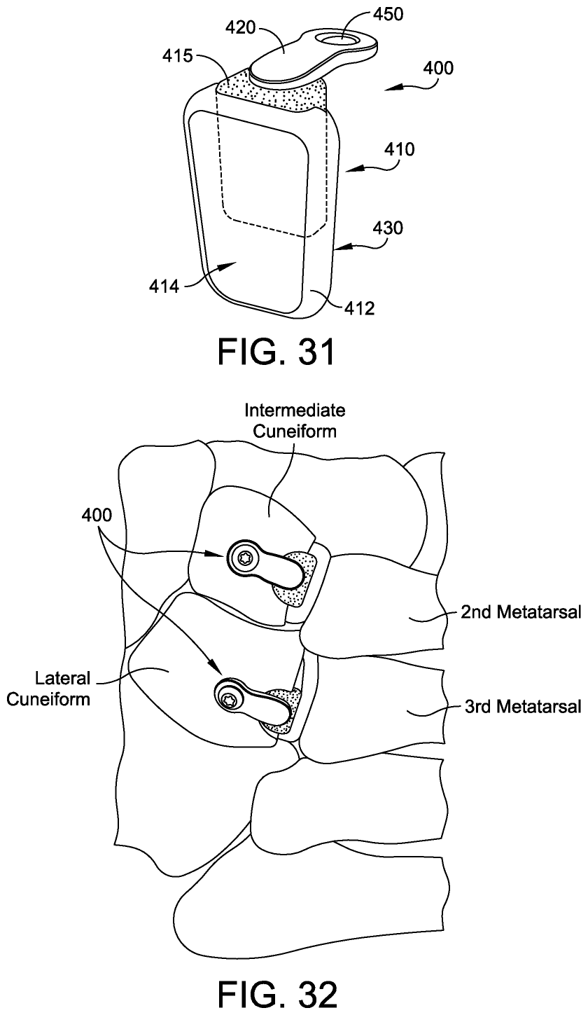

Referring to the cross-sectional views of FIGS. 41 and 42, in some embodiments, the bone plate 420 is formed of a solid metal and comprises a tapered stem 427 that is configured to be inserted into the tapered hole 460 in the porous material portion 415. The tapered stem 427 and the tapered hole 460 cooperate to urge the bone-contacting surface 430 of the implant 400 toward the bone when the implant 400 is inserted into the joint. Referring to FIGS. 31, 40-41, the bone plate 420 comprises at least one screw hole 450 for receiving a bone screw S.

The porous material for the porous material portion 415 can be the same material as the porous material portion 115 of the implant 100 discussed above.

In some embodiments, the hydrogel portion 412 is bonded to the porous material portion 415 by having some hydrogel material infiltrating into pores in a portion of the porous material portion. The main portion 410 comprising the hydrogel portion 412 and the porous material portion 415 can be formed by an appropriate process such as an injection molding or open cavity molding process as described above in connection with the implant embodiment 100.

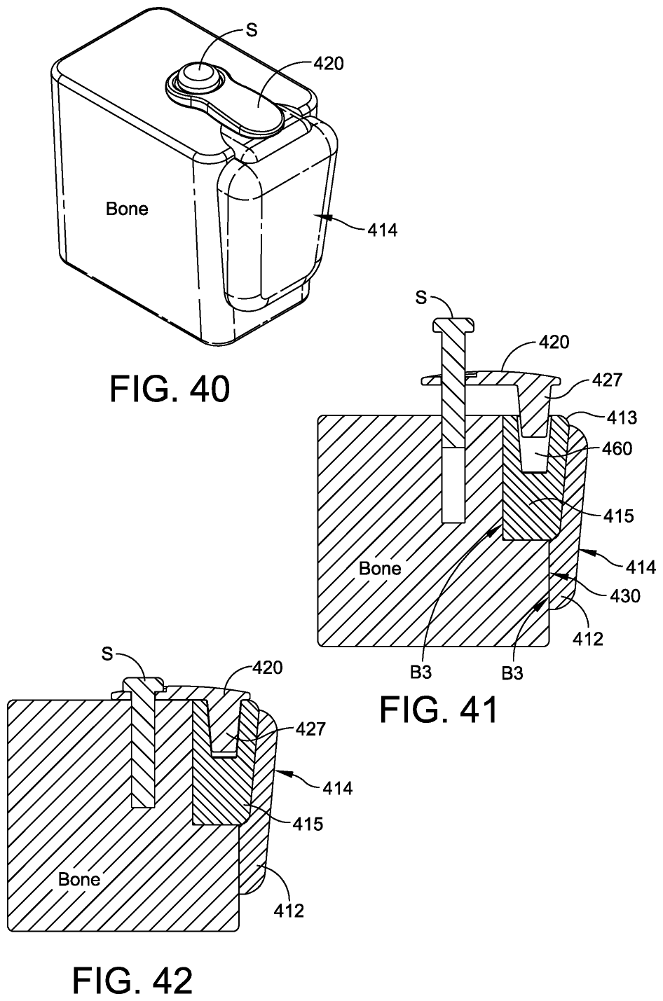

FIGS. 40-42 are illustrated with the bone of a joint in which the implant 400 is secured. A portion of the bone immediately surrounding the implant 400 is illustrated conceptually as a box-like volume Bone for illustration purposes. FIGS. 41 and 42 show how the tapered stem 427 of the bone plate 420 and the tapered hole 460 of the porous material portion 415 engage each other and cooperate to urge the bone-contacting surface 430 of the implant 400 toward the bone when the implant 400 is inserted into the joint. FIG. 41 shows the implant 400 positioned in place in the Bone. The bone-contacting surfaces 430 are contacting the prepared bone surface B3. The tapered stem 427 of the bone plate 420 is partially inserted into the mating tapered hole 460 in the porous material portion 415 and a bone screw S is placed through the screw hole 450 in the bone plate 420 and starting to engage the pre-drilled hole in the Bone. FIG. 42 shows the implant 400 where the bone screw S is fully screwed into the Bone and has secured the bone plate 420 to the Bone. With the bone plate 420 in its fully-seated position, the tapered stem 427 is fully inserted into the tapered hole 460. The tapered surface of the tapered stem 427 pushes against the sidewall of the tapered hole 460 as the tapered stem 427 reaches its fully-seated position and securely holds the main portion 410 of the implant 400 in place.

In some embodiments, the step of preparing the damaged portion of the articulating surface of the joint can comprise cutting into the articulating surface of the distal end of the bone forming the joint with a cutting tool such as a rotating burr 70 to remove a portion of the articulating surface and form the desired resected surfaces B3, B4 (see FIG. 29) that forms at least a portion of the contour of the resected surface necessary for the implant 400.

In some embodiments, the contour of the bone-contacting surface 430 of the implant comprises a flat portion and a protruding portion.

In some embodiments, the step of preparing the damaged portion of the articulating surface of the joint comprises cutting into the articulating surface of the joint with a bone saw 60 to remove a portion of the articulating surface and form a flat resected surface B3 that forms at least a portion of the contour of the resected surface.

In some embodiments, the step of preparing the damaged portion of the articulating surface of the joint comprises cutting into the articulating surface of the joint with a burr 70 to form a half-cylinder shaped surface B4 that matches the contour of the protrusion in the bone-contacting surface 430 of the implant 400.

In some embodiments, the step of preparing the damaged portion of the articulating surface of the joint further comprising cutting into the flat resected surface B3 of the articulating surface with a burr 70 to form a half-cylinder shaped surface B4 that matches the contour of the protrusion in the bone-contacting surface 430 of the implant 400.

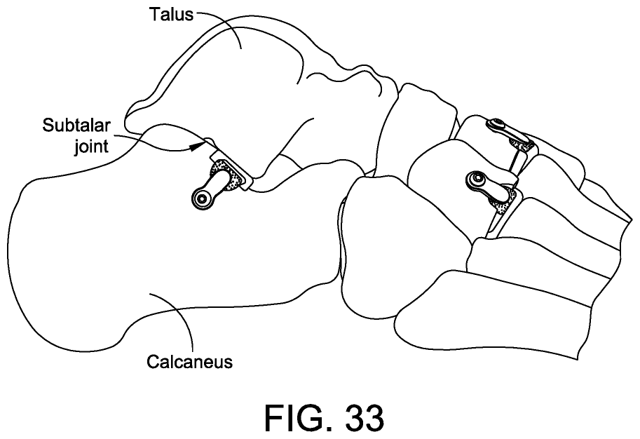

FIGS. 31-33 show the hydrogel implant 400 implanted into some of the mid-foot joints.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

We claim:

1. A method for repairing a damaged portion of an articulating surface of a joint, comprising:
providing an implant that comprises:
a main portion configured for inserting into a joint, wherein the main portion comprises:
a porous material portion having a first bone-engaging surface having a first contour; and
a hydrogel portion that is bonded to the porous material portion and forming an articulating surface opposite from the first bone-engaging surface; and
a bone plate portion configured for securing the implant to a bone that forms the joint;
wherein, the main portion having a leading end and a trailing end, wherein the leading end is configured for being inserted into the joint;
wherein the bone plate portion is integrally formed with the porous material portion and extends from the trailing end, forming a second bone-engaging surface that is also formed of the porous material and extends from the first bone-engaging surface in a direction opposite from the articulating surface at an angle with respect to the first bone-engaging surface, wherein the second bone-engaging surface has a second contour;
wherein the bone plate portion comprises a solid metal portion that forms all exterior surfaces of the bone plate portion except for the second bone-engaging surface; and
wherein the bone plate portion has at least one screw hole for receiving a bone screw;
preparing the damaged portion of the articulating surface of a bone in the joint to have two resected surfaces, one resected surface with a contour that matches the first contour of the first bone-contacting surface, and the second resected surface with a contour that matches the second contour of the second bone-contacting surface of the implant; and
implanting the implant into the joint.

2. The method of claim 1, wherein the step of preparing the damaged portion of the articulating surface of the joint comprises:
cutting into the articulating surface of the joint with a bone saw at a first angle to remove a portion of the articulating surface and form a first resected surface that has a contour that matches the first contour of the first-bone engaging surface; and then
cutting into the articulating surface of the joint with a bone saw at a second angle to remove a portion of the articulating surface and form a second resected surface that has a contour that matches the second contour of the second-bone engaging surface.

3. The method of claim 2, wherein the first resected surface and the second resected surfaces are flat surfaces.

4. The method of claim 1, wherein the step of preparing the damaged portion of the articulating surface of the joint comprises:
cutting into the articulating surface of the joint with a bone saw at a first angle to remove a portion of the articulating surface and form a first resected surface that has a contour that matches the second contour of the second-bone engaging surface; and then
cutting into the articulating surface of the joint with a bone saw at a second angle to remove a portion of the articulating surface and form a second resected surface that has a contour that matches the first contour of the first-bone engaging surface.

5. The method of claim 1, wherein the step of implanting the implant into the joint comprises securing the implant to the two resected surfaces of the bone.

6. A method for repairing a damaged portion of an articulating surface of a joint, comprising:
providing an implant that comprises:
a main portion configured for inserting into a joint, wherein the main portion comprises:
a hydrogel portion forming a bone-contacting surface and an articulating surface opposite from the bone-contacting surface, the bone-contacting surface having a contour;
wherein, the main portion having a leading end and a trailing end, wherein the leading end is configured for being inserted into the joint; and
a bone plate portion configured for securing the implant to a bone that forms the joint; wherein the bone plate portion comprises:
a first part having a perforated structure that is embedded in the hydrogel portion; and
a second part that is not embedded in the hydrogel portion and extending from the trailing end in a direction opposite from the articulating surface at an angle ≤160° but ≥80° with respect to the bone-contacting surface;
wherein the second part has at least one screw hole for receiving a bone screw;
preparing the damaged portion of the articulating surface of the joint to have a resected surface with a contour that matches the contour of the bone-contacting surface of the implant; and
implanting the implant into the joint.

7. The method of claim 5, wherein the step of preparing the damaged portion of the articulating surface of the joint comprises cutting into the articulating surface of the joint with a bone saw to remove a portion of the articulating surface and form a flat resected surface that forms at least a portion of the contour of the resected surface.

8. A method for repairing a damaged portion of an articulating surface of a joint, comprising:
providing an implant that comprises:
a main portion configured for inserting into the joint, wherein the main portion comprises:
a hydrogel portion forming a bone-contacting surface and an articulating surface opposite from the bone-contacting surface;
wherein the bone-contacting surface has a contour that comprises a protruding part that has a half-cylinder contour;
wherein, the main portion having a leading end and a trailing end, wherein the leading end is configured for being inserted into the joint; and
a bone plate portion configured for securing the implant to a bone that forms the joint; wherein the bone plate portion comprises:
a first part having a perforated structure that is embedded in the protruding part of the hydrogel portion; and
a second part that is not embedded in the protruding part of the hydrogel portion and extending from the trailing end in a direction opposite from the articulating surface at an angle ≤160° but ≥80° with respect to the bone-contacting surface;
wherein the second part has at least one screw hole for receiving a bone screw;
preparing the damaged portion of the articulating surface of the joint to have a resected surface with a contour that matches the contour of the bone-contacting surface of the implant; and
implanting the implant into the joint.

9. The method of claim 8, wherein the step of preparing the damaged portion of the articulating surface of the joint comprises cutting into the articulating surface of the joint with a bone saw to remove a portion of the articulating surface and form a flat resected surface that forms at least a portion of the contour of the resected surface.

10. The method of claim 8, wherein the step of preparing the damaged portion of the articulating surface of the joint comprises cutting into the articulating surface of the joint with a burr to form a half-cylinder shaped surface that matches the contour of the protrusion in the bone-contacting surface of the implant.

11. The method of claim 9, further comprising cutting into the flat resected surface of the articulating surface with a burr to form a half-cylinder shaped surface that matches the contour of the protrusion in the bone-contacting surface of the implant.

12. A method for repairing a damaged portion of an articulating surface of a joint, comprising:
providing an implant that comprises:
a main portion configured for inserting into the joint and comprising a leading end, a trailing end, an articulating surface and a bone-contacting surface extending between the leading end and the trailing end, wherein the bone-contacting surface has a contour, wherein the leading end is configured for being inserted into the joint, wherein the main portion further comprises:
a hydrogel portion forming the articulating surface and the bone-contacting surface opposite from the articulating surface; and
a porous material portion bonded to the hydrogel portion and forming a protruding part having a half-cylinder shape that extends from the trailing end and partially towards the leading end, the protruding part forming a portion of the bone-contacting surface;
wherein the porous material portion comprises a tapered hole at the trailing end; and
a bone plate configured for securing the implant to a bone that forms the joint;
wherein the bone plate is formed of a solid metal;
wherein the bone plate comprises a tapered stem that is configured to be inserted into the tapered hole in the porous material portion, whereby the tapered stem and the tapered hole cooperate to urge the bone-contacting surface of the implant toward the bone when the implant is inserted into the joint; and
wherein the bone plate has at least one screw hole for receiving a bone screw;
preparing the damaged portion of the articulating surface of the joint to have a resected surface with a contour that matches the contour of the bone-contacting surface of the implant; and
implanting the implant into the joint.

13. The method of claim 12, wherein the contour of the bone-contacting surface of the implant comprises a flat portion and a protruding portion.

14. The method of claim 12, wherein the step of preparing the damaged portion of the articulating surface of the joint comprises cutting into the articulating surface of the joint with a bone saw to remove a portion of the articulating surface and form a flat resected surface that forms at least a portion of the contour of the resected surface.

15. The method of claim 12, wherein the step of preparing the damaged portion of the articulating surface of the joint comprises cutting into the articulating surface of the joint with a burr to form a half-cylinder shaped surface that matches the contour of the protrusion in the bone-contacting surface of the implant.

16. The method of claim 15, further comprising cutting into the flat resected surface of the articulating surface with a burr to form a half-cylinder shaped surface that matches the contour of the protrusion in the bone-contacting surface of the implant.
